(19) United States
(12) Patent Application Publication
(10) Pub. No.: US 2023/0284946 A1
(43) Pub. Date: Sep. 14, 2023

(54) Small Sample Collection and Dispensing Device for Use with Luer Lock Access Device and Point-of-Care Diagnostics

(71) Applicant: Becton, Dickinson and Company, Franklin Lakes, NJ (US)

(72) Inventors: Megan S. Scherich, Salt Lake City, UT (US); Shaun Staley, Murray, UT (US); Yiping Ma, Layton, UT (US)

(21) Appl. No.: 18/118,449

(22) Filed: Mar. 7, 2023

Related U.S. Application Data
(60) Provisional application No. 63/317,669, filed on Mar. 8, 2022.

(51) Int. Cl. A61B 5/15; A61B 5/153

(52) U.S. Cl. CPC A61B 5/150992; A61B 5/15003; A61B 5/153

(57) ABSTRACT

A point-of-care blood collection and dispensing device for use with a luer lock access device, the point-of-care collection and dispensing device including a distal engagement portion, wherein at least a part of the distal engagement portion is configured to engage a needle of the luer lock access device, and a sidewall portion, wherein at least part of the sidewall portion is formed of a flexible material capable of being compressed. The device also includes a fluid chamber configured to hold a blood sample, the fluid chamber bound at least partially by the distal engagement portion and the sidewall portion, as well as an opening, wherein the opening is sized and configured to dispense a small volume of the blood sample held within the fluid chamber when the sidewall portion is compressed.

### CROSS-REFERENCE TO RELATED APPLICATION

The present application claims priority to U.S. Provisional Application Ser. No. 63/317,669, entitled “Small Sample Collection and Dispensing Device for Use with Luer Lock Access Device and Point-of-Care Diagnostics”, filed Mar. 8, 2022, the entire disclosure of which is hereby incorporated by reference in its entirety.

### BACKGROUND OF THE INVENTION

### Field of the Invention

The present disclosure generally relates to blood collection and dispensing devices and related assemblies, systems, and methods for use in blood collection via a peripheral intravenous catheter (PIVC). The blood collection and dispensing devices are configured to be usable with a luer lock access device coupled to the PIVC for point-of-care (PoC) diagnostics.

### Description of Related Art

A catheter is commonly used to infuse fluids into vasculature of a patient. For example, the catheter may be used for infusing normal saline solution, various medicaments, or total parenteral nutrition. In some instances, catheter may be an over-the-needle peripheral intravenous catheter (PIVC).

In addition to infusion, PIVCs may also be used for withdrawing blood from the patient, although they are not typically designed and optimized for such purposes. In view of various challenges related to blood extraction from PIVCs, fluid transfer devices have been developed to mitigate the possibility of catheter collapse, reduced blood flow due to debris built up on or within the catheter, etc. One such device, PIVO™ from Velano Vascular, Inc., is configured as a single-use device which temporarily attaches to a PIVC to draw a blood sample. Using an existing peripheral intravenous line as a conduit to the vasculature, the PIVO™ device advances a flexible, internal flow tube through the PIVC, beyond the catheter tip, and into the vein to collect a blood sample. Once blood collection is complete, the flow tube is retracted, and the device is removed from the PIVC and discarded.

For blood draws using existing vascular access, the PIVC may be coupled to a blood collection device such as, e.g., a BD VACUTAINER® blood collection tube in order to collect a blood sample from the patient via the PIVC. However, typical blood collection tubes are generally used to take larger volume blood samples and require the use of a separate instrument to collect and dispense smaller amounts of blood for testing purposes. Such a multi-step and multi-component process to collect and dispense blood samples is not ideal for use with point-of-care (PoC) diagnostic testing, which is an increasingly popular testing method that utilizes portable testing systems capable of quickly delivering results using small volume blood samples (e.g., glucose testing). Often, small volume blood samples for use with PoC testing systems are collected via finger pricks, but repeated finger pricks may be uncomfortable for the patient. Accordingly, there is a desire to utilize existing vascular access via the PIVC to collect small volume blood samples for efficient use with PoC testing systems.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In accordance with an aspect of the present disclosure, a point-of-care blood collection and dispensing device for use with a luer lock access device is provided, the device including a distal engagement portion, wherein at least a part of the distal engagement portion is configured to engage a needle of the luer lock access device, and a sidewall portion, wherein at least part of the sidewall portion is formed of a flexible material capable of being compressed. The device also may include a fluid chamber configured to hold a blood sample, the fluid chamber bound at least partially by the distal engagement portion and the sidewall portion, and an opening, wherein the opening is sized and configured to dispense a small volume of the blood sample held within the fluid chamber when the sidewall portion is compressed.

In some embodiments, the device further includes a stopper member, wherein the stopper member is configured to be pierced by the needle of the luer lock access device.

In some embodiments, the stopper member is positioned within the distal engagement portion.

In some embodiments, the stopper member is offset from a central axis of the point-of care blood collection and dispensing device within the distal engagement portion.

In some embodiments, the opening is formed within a valve.

In some embodiments, the valve is a one-way valve.

In some embodiments, the one-way valve is positioned on a proximal end portion of the point-of-care blood collection and dispensing device opposite the distal engagement portion.

In some embodiments, the one-way valve is positioned within the distal engagement portion and is offset from a central axis of the point-of care blood collection and dispensing device.

In some embodiments, the sidewall portion includes an indented portion configured to allow compression of the sidewall portion.

In some embodiments, at least part of the sidewall portion is formed of an elastomeric material.

In some embodiments, the device further includes a proximal vent portion positioned opposite the distal engagement portion, wherein the proximal vent portion includes a hydrophobic vent material.

In accordance with another aspect of the present disclosure, a small sample blood collection system for use with point-of-care diagnostic testing devices is provided, the system including a luer lock access device having a luer lock access hub configured to couple of the luer lock access device to an intermediate device for venous access to a patient, a needle fluidly coupled to the luer lock access hub, and a holder at least partially surrounding the needle and configured to hold a blood collection device. The system also includes a point-of-care blood collection and dispensing device having a distal engagement portion, wherein at least a part of the distal engagement portion is configured to engage the needle of the luer lock access device, a sidewall portion, wherein at least part of the sidewall portion is formed of a flexible material capable of being compressed, a fluid chamber configured to hold a blood sample, the fluid chamber bound at least partially by the distal engagement portion and the sidewall portion, and an opening, wherein the opening is sized and configured to dispense a small volume of the blood sample held within the fluid chamber when the sidewall portion is compressed.

In some embodiments, the luer lock access device further includes a sheath at least partially surrounding the needle.

In some embodiments, the point-of-care blood collection and dispensing device further including a stopper member configured to be pierced by the needle of the luer lock access device.

In some embodiments, the stopper member is offset from a central axis of the point-of care blood collection and dispensing device within the distal engagement portion, and further wherein the needle of the luer lock access device is correspondingly offset from a central axis of the luer lock access device.

In some embodiments, the opening of the point-of-care blood collection and dispensing device is formed within a one-way valve.

In some embodiments, the one-way valve is positioned within the distal engagement portion of the point-of-care blood collection and dispensing device and is offset from a central axis of the point-of care blood collection and dispensing device.

In some embodiments, at least part of the sidewall portion of the point-of-care blood collection and dispensing device is formed of an elastomeric material.

In accordance with another aspect of the present disclosure, a method of small sample blood collection and dispensing is provided, the method including providing a luer lock access device having a luer lock access hub configured to couple of the luer lock access device to an intermediate device for venous access to a patient, a needle fluidly coupled to the luer lock access hub, and a holder at least partially surrounding the needle and configured to hold a blood collection device. The method also includes providing a point-of-care blood collection and dispensing device having a distal engagement portion, wherein at least a part of the distal engagement portion is configured to engage the needle of the luer lock access device, a sidewall portion, wherein at least part of the sidewall portion is formed of a flexible material capable of being compressed, a fluid chamber configured to hold a blood sample, the fluid chamber bound at least partially by the distal engagement portion and the sidewall portion, and an opening, wherein the opening is sized and configured to dispense a small volume of the blood sample held within the fluid chamber when the sidewall portion is compressed. The method also includes distally directing the point-of-care blood collection and dispensing device toward the luer lock access device such that the needle of the luer lock access device punctures at least a portion of the distal engagement portion of the point-of-care blood collection and dispensing device, and collecting a blood sample within the fluid chamber of the point-of-care blood collection and dispensing device.

In some embodiments, the method also includes removing the point-of-care blood collection and dispensing device from engagement with the needle of the luer lock access device, positioning the point-of-care blood collection and dispensing device relative to a point-of-care diagnostic testing device, and compressing at least a portion of the sidewall portion of the point-of-care blood collection and dispensing device so as to dispense a portion of the blood sample collected within the fluid chamber into or onto the point-of-care diagnostic testing device.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is a side cross-sectional view of a luer lock access device in accordance with an aspect of the present disclosure;

FIG. 2 is a side cross-sectional view of a blood collection and dispensing device for use with the luer lock access device of FIG. 1 in accordance with an aspect of the present disclosure;

FIG. 3 is a side cross-sectional view of a blood collection and dispensing device in accordance with another aspect of the present disclosure;

FIG. 4 is a side cross-sectional view of a blood collection and dispensing device in accordance with another aspect of the present disclosure;

FIG. 5 is a side cross-sectional view of a blood collection and dispensing device in accordance with another aspect of the present disclosure; and

FIG. 6 is an isometric view of the blood collection and dispensing device of FIG. 5 in use with a PoC testing cartridge in accordance with an aspect of the present disclosure.

### DESCRIPTION OF EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood collection and dispensing device in preparation or during use. Similarly, in this application, the terms “in the distal direction” and “distally” mean in the direction toward the luer lock access device, and the terms “in the proximal direction” and “proximally” mean in the direction opposite the direction of the luer lock access device.

While not shown or described herein, it is to be understood that the blood collection and dispensing devices described below may be utilized for blood draw from any suitable catheter system such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly.

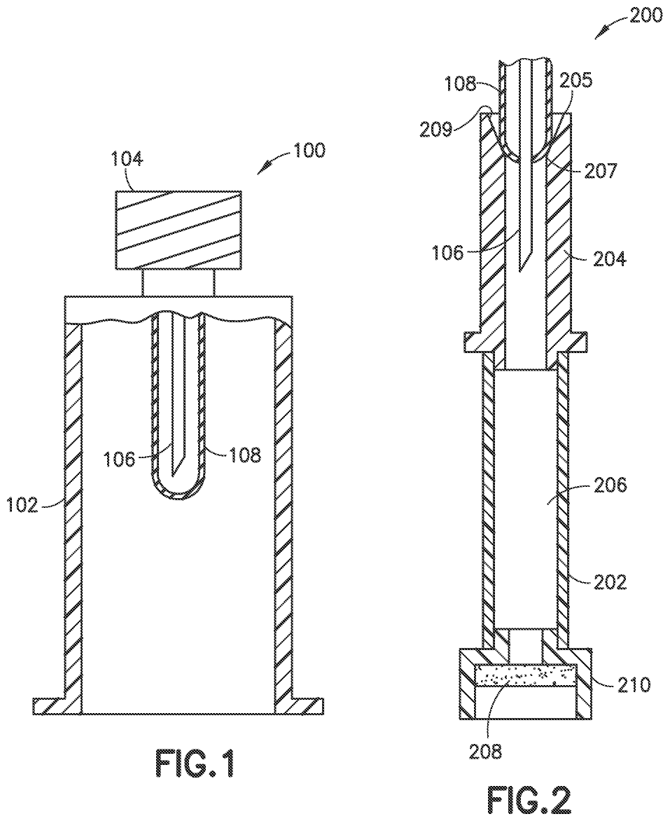

Referring to FIG. 1, a luer lock access device 100 in accordance with an aspect of the present disclosure is illustrated. In some embodiments, the luer lock access device 100 may be coupled to a PIVC (not shown) via any suitable blood collection adapter or other intermediate device. As shown, the luer lock access device 100 is configured to receive a blood collection device such as, e.g., a BD VACUTAINER® blood collection tube. The luer lock access device 100 includes a luer lock hub 104. In some embodiments, the luer lock hub 104 may have internal threads (not shown) capable of engaging a proximal coupling portion of a blood collection adapter or other device to the luer lock access device 100 thereto.

The luer lock access device 100 further includes a needle 106 in fluid communication with the luer lock hub 104 so as to allow fluids to pass from the blood collection adapter (or other device) to a blood collection device. The needle 106 may be substantially surrounded by a sheath 108 to protect from needle stick injuries. The sheath 108 may be formed of rubber or any other appropriate material. Furthermore, the luer lock access device 100 may include a holder 102, which is sized and configured to accommodate a blood collection device such as, e.g., a BD VACUTAINER® blood collection tube. In response to the blood collection device pushing the sheath 108 distally towards the luer lock hub 104, the needle 106 may pierce the sheath 108 and the sharp proximal tip of the needle 106 may be inserted into the blood collection device so as to receive a blood sample via the blood collection adapter or other intermediate device.

As noted above, the holder 102 of luer lock access device 100 may be sized and configured to accommodate a blood collection device such as, e.g., a BD VACUTAINER® blood collection tube. However, such a blood collection device is not generally utilized with point-of-care (PoC) diagnostic testing devices, as a separate collection instrument must be employed to collect and dispense the small samples required by PoC diagnostic testing devices.

Thus, in accordance with an aspect of the present disclosure, and as is shown in FIG. 2, a PoC blood collection/dispensing device 200 for use with the luer lock access device 100 is provided. PoC blood collection/dispensing device 200 includes a flexible sidewall portion 202, a distal introducer portion 204, and a proximal vent portion 210. The flexible sidewall portion 202 substantially surrounds a fluid chamber 206, which is sized and configured to hold a small volume blood sample obtained via venous pressure through the luer lock access device 100. The proximal vent portion 210 may include a hydrophobic vent material 208 to enable blood to be drawn into the fluid chamber by venous pressure alone.

Referring still to FIG. 2, the distal introducer portion 204 includes a distal end 205. In some embodiments, the distal introducer portion 204 may include an angled radial surface 209 extending inward from the distal end 205, with the angled radial surface 209 configured to contact sheath 108 surrounding the needle 106 of the luer lock access device 100. When a user wishes to obtain a blood sample, the angled radial surface of the distal introducer portion 204 may be positioned over a proximal tip of the sheath 108, and the entire PoC blood collection/dispensing device 200 may be pressed proximally, thereby compressing the sheath 108 until punctured by the needle 106, as is illustrated in FIG. 2. In this position, the fluid chamber 206 is fluidly coupled to the needle 106 such that a small volume blood sample can be collected within the PoC blood collection/dispensing device 200.

After a desired blood sample is collected in the fluid chamber 206, the PoC blood collection/dispensing device 200 is moved proximally in order to decouple the distal introducer portion 204 from the sheath 108 and needle 106. The distal introducer portion 204 may include an opening 207 sized and configured such that surface tension alone is sufficient to retain a blood sample within the fluid chamber 206 before dispensing of the sample, thereby eliminating the need for any valve or other closure associated with the distal introducer portion 204. Furthermore, an inner diameter and/or shape of the distal introducer portion 204 may be configured so as to limit the volume and/or speed at which blood is dispensed therethrough, thereby ensuring that an appropriately small sample can be dispensed to a PoC diagnostic testing device in a controlled manner.

In order to dispense the blood sample from within the fluid chamber 206, the flexible sidewall portion 202 of the PoC blood collection/dispensing device 200 can be squeezed or otherwise compressed by the user in order to force droplets of the sample out of the opening 207 of distal introducer portion 204. The flexible sidewall portion 202 may be formed of any appropriate flexible material such as, e.g., a molded elastomeric material, and may have any appropriate durometer and/or thickness in order to achieve a desired force required to flex the surface for controlled dispensing of blood from the distal introducer portion 204. Furthermore, all or some of the flexible sidewall portion 202 may be formed of a translucent or transparent material so as to enable a user to visualize the sample contained therein. In some embodiments, at least a portion of the flexible sidewall portion 202 may include volumetric graduation markings thereon.

As noted above, the PoC blood collection/dispensing device 200 is usable with any appropriate PoC diagnostic testing device including cartridges for systems such as, e.g., an i-STAT handheld blood analyzer from Abbott, test strips for glucose monitors, etc. After dispensing of a desired volume of a blood sample to the PoC diagnostic testing device, the entire PoC blood collection/dispensing device 200 may be discarded in an appropriate medical waste container. Accordingly, the PoC blood collection/dispensing device 200 provides an easy and efficient solution to collecting and dispensing small volume blood samples using existing vascular access via a PIVC.

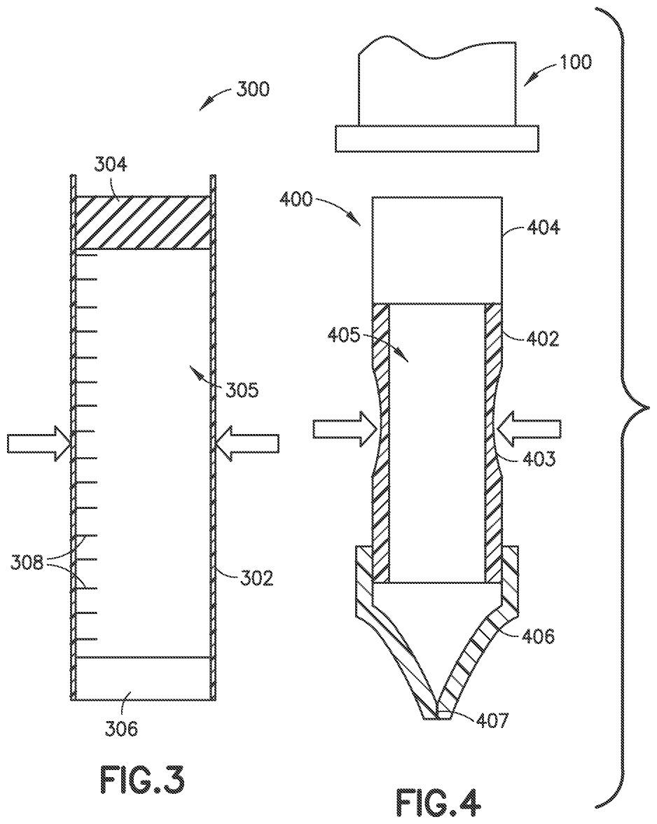

Next, referring to FIG. 3, PoC blood collection/dispensing device 300 in accordance with another aspect of the present disclosure is shown. While not shown, similar to PoC blood collection/dispensing device 200 described above with respect to FIG. 2, PoC blood collection/dispensing device 300 is configured to be usable with a luer lock access device in order to collect a small volume blood sample via existing vascular access.

The PoC blood collection/dispensing device 300 includes a sidewall portion 302 that is at least partially compressible, a stopper member 304, and a valve member 306. The sidewall portion 302, stopper member 304, and valve member 306 bound a fluid chamber 305 sized and configured to hold a small volume blood sample. Volumetric graduation markings 308 may be provided on at least part of the sidewall portion 302.

Stopper member 304 may be formed of any appropriate material capable of engaging with the needle of a luer lock access device such as, e.g., rubber. Valve member 306 may be selectively opened and closed by a user in order to control the collection of a blood sample within the PoC blood collection/dispensing device 300.

To initiate a blood draw, the user may press the PoC blood collection/dispensing device 300 distally such that the stopper member 304 is fully pierced by a needle of a luer lock access device, thereby fluidly coupling the fluid chamber 305 with the luer lock access device. Then, to start blood draw via venous pressure, the valve member 306 may be opened, thus allowing blood to begin to fill the fluid chamber 305. Once a desired volume of blood is collected within the fluid chamber 305, the valve member 306 may be closed and the stopper member 304 may be disengaged from the luer lock access device.

To dispense the blood sample into a PoC diagnostic testing device, the valve member 306 may be opened, and at least a portion of the sidewall portion 302 may be squeezed or otherwise compressed by the user, thereby forcing a small amount of the blood sample out of the fluid chamber 305 via the valve member 306. The valve member 306 may be configured so as to limit the volume and/or speed at which blood is dispensed therethrough. Furthermore, at least the compressible region of the sidewall portion 302 may be formed of any appropriate flexible material such as, e.g., a molded elastomeric material, and may have any appropriate durometer and/or thickness in order to achieve a desired force required to flex the surface for controlled dispensing of blood from the valve member 306.

Referring now to FIG. 4, a PoC blood collection/dispensing device 400 in accordance with another aspect of the present disclosure is illustrated. As shown, PoC blood collection/dispensing device 400 is configured to be usable with the luer lock access device 100 in order to collect a small volume blood sample via existing vascular access.

The PoC blood collection/dispensing device 400 includes a sidewall portion 402 having an indented portion 403. The indented portion 403 is configured such that the wall thickness of sidewall portion 402 is thinned, thereby providing for a compressible weak point in the sidewall portion 402. In some embodiments, the indented portion 403 may extend fully around the sidewall portion 402. In other embodiments, the indented portion 403 may extend only partially around the sidewall portion 402.

The PoC blood collection/dispensing device 400 further includes a stopper member 404 and a one-way valve member 406. The sidewall portion 402, stopper member 404, and one-way valve member 406 bound a fluid chamber 405 sized and configured to hold a blood sample. Although not shown in FIG. 4, volumetric graduation markings may be provided on at least part of the sidewall portion 402.

Stopper member 404 may be at least partially formed of any appropriate material capable of engaging with the needle of a luer lock access device such as, e.g., rubber. One-way valve member 406 includes an opening 407 which may open under fluid force in the proximal direction in order to dispense a small volume blood sample at a desired rate.

To initiate a blood draw, the user may press the PoC blood collection/dispensing device 400 distally such that at least a portion of the stopper member 404 is fully pierced by a needle of a luer lock access device 100, thereby fluidly coupling the fluid chamber 405 with the luer lock access device 100. Once a desired volume of blood is collected within the fluid chamber 405, the stopper member 404 may be disengaged from the luer lock access device 100.

To dispense the blood sample into a PoC diagnostic testing device, the indented portion 403 of the sidewall portion 402 may be squeezed or otherwise compressed by the user, thereby forcing a small amount of the blood sample out of the fluid chamber 305 via the opening 407 of the one-way valve member 406. The one-way valve member 406 may be configured so as to limit the volume and/or speed at which blood is dispensed therethrough. Furthermore, at least the indented portion 403 of the sidewall portion 402 may be formed of any appropriate flexible material such as, e.g., a molded elastomeric material, and may have any appropriate durometer and/or thickness in order to achieve a desired force required to flex the surface for controlled dispensing of blood from the one-way valve member 406.

Unlike the PoC blood collection/dispensing device 200, 300 described above with respect to FIGS. 2 and 3, the PoC blood collection/dispensing device 400 may be configured to have a form factor similar to that of a conventional blood collection device such as, e.g., a BD VACUTAINER® blood collection tube. Accordingly, the PoC blood collection/dispensing device 400 may be sized and configured to fit securely within the holder of luer lock access device 100 when engaging the needle of the luer lock access device 100.

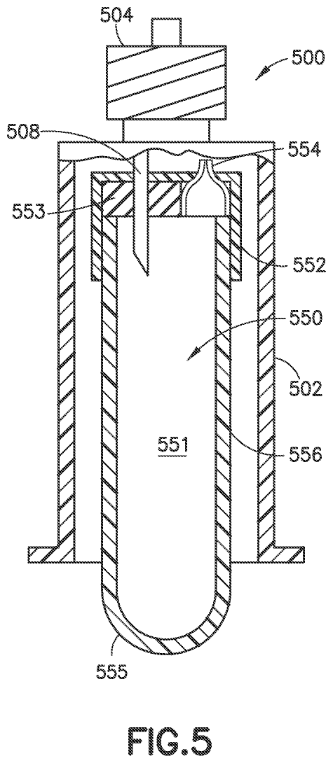

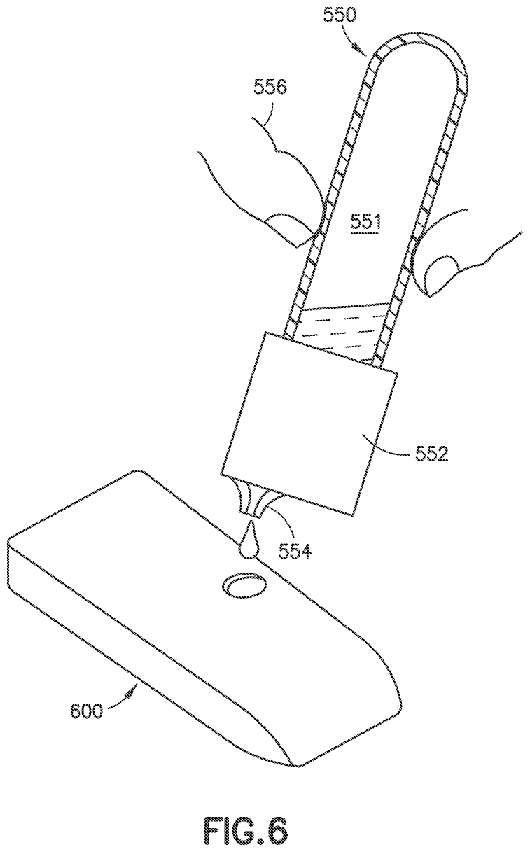

Next, referring to FIGS. 5 and 6, a luer lock access device 500 and PoC blood collection/dispensing device 550 in accordance with another aspect of the present disclosure are illustrated.

In some embodiments, the luer lock access device 500 may be coupled to a PIVC (not shown) via any suitable blood collection adapter or other intermediate device. The luer lock access device 500 includes a luer lock hub 504. In some embodiments, the luer lock hub 504 may have internal threads capable of engaging a proximal coupling portion of a blood collection adapter or other device to the luer lock access device 500 thereto. The luer lock access device 500 further includes a needle 508 in fluid communication with the luer lock hub 504 so as to allow fluids to pass from the blood collection adapter (or other device) to a blood collection device. Unlike needle 106 shown and described above with respect to luer lock access device 100, the needle 508 is offset from a central axis of the luer lock hub 504. Furthermore, while not shown in FIG. 5, the needle 508 may be substantially surrounded by a sheath to protect from needle stick injuries. The sheath may be formed of rubber or any other appropriate material. The luer lock access device 500 may also include a holder 502, which may be sized and configured to accommodate a conventional blood collection device such as, e.g., a BD VACUTAINER® blood collection tube.

As shown in FIG. 5, the PoC blood collection/dispensing device 550 is configured to be usable with the luer lock access device 500 in order to collect a small volume blood sample via existing vascular access. The PoC blood collection/dispensing device 550 includes a flexible sidewall portion 556. At least part of the flexible sidewall portion 556 may be formed of any appropriate flexible material such as, e.g., a molded elastomeric material, and may have any appropriate durometer and/or thickness in order to achieve a desired force required to flex the surface for controlled dispensing of blood the PoC blood collection/dispensing device 550. Alternatively, a portion of the flexible sidewall portion 556 may be thinned to enable compression by the user's fingers and/or thumbs. The thinned portion of the flexible sidewall portion 556 may have finger/thumb prints molded therein to provide a visual and/or tactile indicator to the user regarding the location of the compressible portion. Furthermore, all or some of the flexible sidewall portion 556 may be formed of a translucent or transparent material so as to enable a user to visualize the sample contained therein. In some embodiments, at least a portion of the flexible sidewall portion 556 may include volumetric graduation markings thereon.

The PoC blood collection/dispensing device 550 further includes a closed end 555 and a distal engagement portion 552. The distal engagement portion 552 includes both a stopper member 553 and a one-way valve member 554. The stopper member 553 and one-way valve member 554 are each offset from a central axis of the PoC blood collection/dispensing device 550. Thus, unlike the PoC blood collection/dispensing device 400 described above with respect to FIG. 4, both the stopper member 553 and one-way valve member 554 are arranged on a distal portion of the PoC blood collection/dispensing device 550.

The flexible sidewall portion 556, distal engagement portion 552, and closed end 555 bound a fluid chamber 551 sized and configured to hold a blood sample. Although not shown in FIGS. 5 and 6, volumetric graduation markings may be provided on at least part of the sidewall portion 556. Stopper member 553 may be at least partially formed of any appropriate material capable of engaging with the needle 508 of a luer lock access device 500 such as, e.g., rubber. One-way valve member 554 includes an opening which may open under fluid force in the distal direction in order to dispense a small volume blood sample at a desired rate.

To initiate a blood draw, the user may press the PoC blood collection/dispensing device 550 distally such that at least a portion of the offset stopper member 553 is fully pierced by the correspondingly offset needle 508 of a luer lock access device 500, thereby fluidly coupling the fluid chamber 551 with the luer lock access device 500. Once a desired volume of blood is collected within the fluid chamber 551, the stopper member 553 may be disengaged from the luer lock access device 500.

Next, referring to FIG. 6, to dispense the blood sample into a PoC diagnostic testing device (e.g., cartridge 600), at least a portion of the flexible sidewall portion 556 may be squeezed or otherwise compressed by the user, thereby forcing a small amount of the blood sample out of the fluid chamber 551 via the opening of the one-way valve member 554 provided in the distal engagement portion 552. The one-way valve member 554 may be configured so as to limit the volume and/or speed at which blood is dispensed therethrough.

Similar to the PoC blood collection/dispensing device 400 described above with respect to FIG. 4, the PoC blood collection/dispensing device 550 may be configured to have a form factor similar to that of a conventional blood collection device such as, e.g., a BD VACUTAINER® blood collection tube. Accordingly, the PoC blood collection/dispensing device 550 may be sized and configured to fit securely within the holder 502 of luer lock access device 500 when engaging the needle 508 of the luer lock access device 500.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A point-of-care blood collection and dispensing device for use with a luer lock access device, comprising:
a distal engagement portion, wherein at least a part of the distal engagement portion is configured to engage a needle of the luer lock access device;
a sidewall portion, wherein at least part of the sidewall portion is formed of a flexible material capable of being compressed;
a fluid chamber configured to hold a blood sample, the fluid chamber bound at least partially by the distal engagement portion and the sidewall portion; and
an opening, wherein the opening is sized and configured to dispense a small volume of the blood sample held within the fluid chamber when the sidewall portion is compressed.

2. The point-of-care blood collection and dispensing device of claim 1, further comprising a stopper member, wherein the stopper member is configured to be pierced by the needle of the luer lock access device.

3. The point-of-care blood collection and dispensing device of claim 2, wherein the stopper member is positioned within the distal engagement portion.

4. The point-of-care blood collection and dispensing device of claim 2, wherein the stopper member is offset from a central axis of the point-of care blood collection and dispensing device within the distal engagement portion.

5. The point-of-care blood collection and dispensing device of claim 1, wherein the opening is formed within a valve.

6. The point-of-care blood collection and dispensing device of claim 5, wherein the valve is a one-way valve.

7. The point-of-care blood collection and dispensing device of claim 6, wherein the one-way valve is positioned on a proximal end portion of the point-of-care blood collection and dispensing device opposite the distal engagement portion.

8. The point-of-care blood collection and dispensing device of claim 6, wherein the one-way valve is positioned within the distal engagement portion and is offset from a central axis of the point-of care blood collection and dispensing device.

9. The point-of-care blood collection and dispensing device of claim 1, wherein the sidewall portion comprises an indented portion configured to allow compression of the sidewall portion.

10. The point-of-care blood collection and dispensing device of claim 1, wherein at least part of the sidewall portion is formed of an elastomeric material.

11. The point-of-care blood collection and dispensing device of claim 1, further comprising a proximal vent portion positioned opposite the distal engagement portion, wherein the proximal vent portion comprises a hydrophobic vent material.

12. A small sample blood collection system for use with point-of-care diagnostic testing devices, comprising:
a luer lock access device comprising:
a luer lock access hub configured to couple of the luer lock access device to an intermediate device for venous access to a patient,
a needle fluidly coupled to the luer lock access hub, and
a holder at least partially surrounding the needle and configured to hold a blood collection device; and
a point-of-care blood collection and dispensing device comprising:
a distal engagement portion, wherein at least a part of the distal engagement portion is configured to engage the needle of the luer lock access device,
a sidewall portion, wherein at least part of the sidewall portion is formed of a flexible material capable of being compressed,
a fluid chamber configured to hold a blood sample, the fluid chamber bound at least partially by the distal engagement portion and the sidewall portion, and
an opening, wherein the opening is sized and configured to dispense a small volume of the blood sample held within the fluid chamber when the sidewall portion is compressed.

13. The small sample blood collection system of claim 12, wherein the luer lock access device further comprises a sheath at least partially surrounding the needle.

14. The small sample blood collection system of claim 12, wherein the point-of-care blood collection and dispensing device further comprises a stopper member configured to be pierced by the needle of the luer lock access device.

15. The small sample blood collection system of claim 14, wherein the stopper member is offset from a central axis of the point-of care blood collection and dispensing device within the distal engagement portion, and further wherein the needle of the luer lock access device is correspondingly offset from a central axis of the luer lock access device.

16. The small sample blood collection system of claim 12, wherein the opening of the point-of-care blood collection and dispensing device is formed within a one-way valve.

17. The small sample blood collection system of claim 16, wherein the one-way valve is positioned within the distal engagement portion of the point-of-care blood collection and dispensing device and is offset from a central axis of the point-of care blood collection and dispensing device.

18. The small sample blood collection system of claim 12, wherein at least part of the sidewall portion of the point-of-care blood collection and dispensing device is formed of an elastomeric material.

19. A method of small sample blood collection and dispensing, comprising:
providing a luer lock access device comprising:
a luer lock access hub configured to couple of the luer lock access device to an intermediate device for venous access to a patient,
a needle fluidly coupled to the luer lock access hub, and
a holder at least partially surrounding the needle and configured to hold a blood collection device;
providing a point-of-care blood collection and dispensing device comprising:
a distal engagement portion, wherein at least a part of the distal engagement portion is configured to engage the needle of the luer lock access device,
a sidewall portion, wherein at least part of the sidewall portion is formed of a flexible material capable of being compressed,
a fluid chamber configured to hold a blood sample, the fluid chamber bound at least partially by the distal engagement portion and the sidewall portion, and
an opening, wherein the opening is sized and configured to dispense a small volume of the blood sample held within the fluid chamber when the sidewall portion is compressed;
distally directing the point-of-care blood collection and dispensing device toward the luer lock access device such that the needle of the luer lock access device punctures at least a portion of the distal engagement portion of the point-of-care blood collection and dispensing device; and
collecting a blood sample within the fluid chamber of the point-of-care blood collection and dispensing device.

20. The method of claim 19, further comprising:
removing the point-of-care blood collection and dispensing device from engagement with the needle of the luer lock access device;
positioning the point-of-care blood collection and dispensing device relative to a point-of-care diagnostic testing device; and
compressing at least a portion of the sidewall portion of the point-of-care blood collection and dispensing device so as to dispense a portion of the blood sample collected within the fluid chamber into or onto the point-of-care diagnostic testing device.
